(19) United States
(12) Patent Application Publication
(10) Pub. No.: US 2024/0293446 A1
(43) Pub. Date: Sep. 5, 2024

(54) TCM THROAT TEA METHOD AND COMPOSITION

(71) Applicant: Maishen Inc., Santa Clara, CA (US)

(72) Inventors: Dalong Zhang, Santa Clara, CA (US); Xinghe Chen, Santa Clara, CA (US); Eva Chen, Santa Clara, CA (US)

(73) Assignee: Maishen Inc., Santa Clara, CA (US)

(21) Appl. No.: 18/661,506

(22) Filed: May 10, 2024

(51) Int. Cl. A61K 33/06; A23F 3/34; A61K 35/02; A61K 36/484; A61K 36/71; A61K 36/736; A61K 36/78; A61K 36/8966; A61K 36/899; A61K 36/9068

(52) U.S. Cl. CPC A61K 33/06; A23F 3/34; A61K 35/02; A61K 36/484; A61K 36/71; A61K 36/736; A61K 36/78; A61K 36/8966; A61K 36/899; A61K 36/9068; A61K 2236/15; A61K 2236/17; A61K 2236/37

(57) ABSTRACT

A method and composition to extract of several processed herbal medicinal plants and an herbal tea composition for ingestion which contains the extract. The functional herbal extraction method and composition is used to relieve the symptoms of cough and throat discomfort. This composition creates a synergy between different ingredients that works to suppress cough, yellow or green phlegm, sore throat and specifically to treat hot cough. The composition can include Shi Gao, Hai Fu Shi, Chuan Bei Mu, Tian Zhu Huang, Ban Xia, Yu Xing Cao, Tian Kui Zi, Gan Cao, Tian Nan Xing, Chai Hu, Huang Qin, Zhi Shi, Bai Shao, Chen Pi, Kuan Dong Hua, Zi Wan, Sheng Jiang. The effectiveness of the herbal tea is at least partially dependent on the desired means of preparation and administering the composition.

### FIELD OF THE INVENTION

The present invention relates generally to a cough relief herbal beverage method and composition. Further, the present invention relates to an herbal method and composition that balances the circulation of Qi and blood, balancing the lung cold and lung heat, thereby reducing phlegm and relieves cough, through a unique method of preparing a composition consisting of the Chinese herbal medicine.

### BACKGROUND OF THE INVENTION

Cough is the most common reason for patients to seek medical treatment in the outpatient setting. Cough can be a warning sign of respiratory or non-respiratory diseases. According to previous clinical manifestations, dry cough symptoms generally appear in the late stages of a cold. It is commonly believed that gastroesophageal reflux, post-nasal drip, or asthma can cause chronic cough. In Traditional Chinese Medicine (TCM), when the lungs are attacked by cold or heat, the alveolar mucosal tissue spasms. This leads to spasmodic cough in the bronchus, accompanied by sticky sputum or no sputum, and the symptoms lasts about a week. The current symptoms of dry cough are different from those in the past. Most patients with dry cough have a history of COVID-19 or influenza, and a few have vaccine sequelae. The duration of dry cough for those suffering from COVID-19 or influenza usually lasts for more than 3-4 weeks, and some patients may experience dry cough symptoms for months or even years. This kind of dry cough is called viral dry cough. The main reason why the currently popular dry cough symptoms cannot be treated with traditional cough syrups is that the causes of previous coughs and the currently popular dry coughs are different, and the classification of dry coughs is different.

Covid-19 virus often remains dormant in the body, and in some cases, it is possible that Covid latent viruses are reactivated, causing Covid symptoms amongst patients. When these viruses remain dormant in the patient's body, and the patient's immunity declines or the weather changes, these viruses will become active. At the baseline, the patient has constant dry cough and rarely produces sputum; and if the patient's immunity begins to increase, the anti-viral ability begins to increase, and the virus is in a dormant state, tentatively determined to be below the 60-point level, then the symptoms of dry cough will weaken or even disappear. In this way, dry cough symptoms recur, and the patient is constantly disturbed.

It is widely recognized in Traditional Chinese Medicine (TCM) that cough is a symptom with which the lung reacts to a form of irritation, which is considered as a ‘lung counterflow qi.’ In TCM, the lungs disperse and regulate Qi. Their proper functioning is dependent on the harmonious relationship with other organs, particularly the spleen and kidneys, which can sometimes be disrupted by external pathogens or internal disharmonies. Phlegm in the lung is created through damp obstruction of the middle jiao, arising from inadequate functioning of the spleen. Phlegm stagnation in the lung leads to failure of lung qi to descend, producing cough, dyspnea, or wheezing, stifling sensations in the chest, and profuse sputum.

As a method to treat cough symptoms, TCM traditionally teaches that clam and snow pear soup can cure cough symptoms but this is not the case. The reason is that the sugar in fruits or desserts will accelerate the reproduction rate of dry cough viruses, and aggravate dry cough. Some customers will choose “Golden Throat” to treat throat pain, but since it contains sugar, the results are inconclusive. Sugar will accelerate the reproduction rate of dry cough viruses and cause throat pain to worsen. If we go back to the theory of Traditional Chinese Medicine (TCM), eating sugar will damage the spleen, the spleen will generate dampness, and the dampness will generate phlegm. This explanation differs from biochemical theory, but the result is the same.

### SUMMARY OF THE INVENTION

This invention generally relates to extracts of several processed herbal medicinal plants and an herbal tea composition for ingestion which contains the extract. The invention's composition is used to relieve the symptoms of cough and throat discomfort. This composition creates a synergy between different ingredients that works to suppress cough, yellow or green phlegm, and sore throat.

The invention is due, in part, to the unexpected finding that administration of the present invention's composition relieves hot cough and dry cough, diagnosable conditions in TCM. TCM diagnose cold, hot, dry, wet coughs by examining patients' pulse speed, tongue coating, color of the sputum, and color of the nasal mucus. The present invention's composition is at least used to relieve symptoms of hot cough and dry cough.

In a preferred embodiment of the present invention, the composition includes Shi Gao, Yu Xing Cao, Chuan Bei Mu, Tian Kui Zi, Tian Zhu Huang, Hai Fu Shi, Gan Cao. The effectiveness of herbal tea is at least partially dependent on the desired means of preparation and administering the composition.

In another preferred embodiment of the present invention, the composition includes Shi Gao, Almonds, Hai Fu Shi, Chuan Bei Mu, Tian Zhu Huang, Ban Xia.

In another preferred embodiment of the present invention, the composition includes Shi Gao, Almonds, Hai Fu Shi, Chuan Bei Mu, Tian Zhu Huang, Ban Xia, Yu Xing Cao, Tian Kui Zi, Gan Cao, Tian Nan Xing, Chai Hu, Huang Qin, Zhi Shi, Bai Shao, Chen Pi, Kuan Dong Hua, Zi Wan, Sheng Jiang.

In one embodiment, the therapeutically effective amount comprises from between about 1 mg to about 14000 mg/day.

In a preferred embodiment of the present invention, the therapeutically effective amount comprises from between about 330 mg to 660 mm/day.

In a preferred embodiment of the present invention, the therapeutically effective amount is a dosage regiment of half of a bottle in the morning, and half of a bottle in the evening, wherein each bottle comprises about 330 mg of the composition.

In a preferred embodiment of the present invention, the therapeutically effective amount is a dosage regiment of one bottle in the morning, and one bottle in the evening, wherein each bottle comprises about 330 mg of the composition.

In one embodiment, the administration to the subject occurs between about 30 minutes to about 1 hour after consuming food.

In another embodiment, the composition is added with equal parts of water and heated over the stovetop or microwave, then administered to the subject.

In another preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the first extract, adding an aqueous solution to the remaining solids of the first extract, heat the aqueous solution containing solids from the extract from about room temperature to about boiling temperature, reduction of the aqueous solution contain the first extract by simmering, filtering the aqueous solution containing solids from the first extract to obtain the second extract, combining both extracts to create a third extract, reducing the third extract with a condensing machine.

In another preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the extract.

In another preferred embodiment of the present invention, the composition is administered in at least one of the following ways: oral, topical, suppository, intravenous, intradermic, intramuscular, and intraperitoneal.

In another preferred embodiment of the present invention, the composition includes at least one member selected from the group consisting of: a tablet, an orally disintegrating tablet, a pill, a dripping pill, a lozenge, a capsule, a cream, a granule, a suppository, and an oral liquid.

In another preferred embodiment of the present invention, the composition consists of a plurality of herbal ingredients prepared through processing methods, extraction methods, and purification methods.

In another preferred embodiment of the present invention, the processing methods include at least one member selected from the group consisting of: washing, steaming, extracting, roasting, herb frying, salt frying, honey frying, wine frying, earth frying, vinegar frying, calcining drying, heating, and grinding.

In another preferred embodiment of the present invention, the extraction methods include at least one member selected from the group consisting of: soaking, heating, steaming, evaporation, compressing, and solvent extraction with at least one of the following: water, alcoholic solvents mixed with water, petroleum ether, hexane, diethyl amine, diethyl ether, cyclohexane, tert-butyl alcohol, isopropanol, Acetonitrile, acetone, ethanol, methyl isobutyl ketone, isobutyl alcohol, 1-propanol, methyl ethyl ketone, 2-butanol, isoamyl alcohol, 1-butanol, diethyl ketone, 1-octanol, p-xylene, m-xylene, toluene, dimethoxyethane, benzene, butyl acetate, 1-chlorobutane, tetrahydrofuran, ethyl acetate, o-xylene, hexamethylphosphorus triamide, 2-ethoxyethyl ether. N,N-dimethylacetamide, diethylene glycol dimethyl ether, N,N-dimethylformamide, 2-methoxyethanol, pyridine, propanoic acid, 2-methoxyethyl acetate, benzonitrile, 1-Methyl-2-pyrrolidinone, hexamethylphosphoramide, 1,4-dioxane, acetic acid, acetic anhydride, dimethyl sulfoxide, chlorobenzene, ethylene glycol, diethylene glycol, propylene carbonate, formic acid, 1,2-dichloroethane, glycerin, carbon disulfide, 1,2-dichlorobenzene, methylene chloride, nitromethane, 2,2,2-trifluoroethanol, chloroform, 1,1,2-trichlorotrifluoroethane, carbon tetrachloride, and tetrachloroethylene.

In another preferred embodiment of the present invention, the purification methods include at least one member selected from the group consisting of: column separation, solvent partition, evaporation, and solvent precipitation.

One objective of the present invention is to provide an herbal composition that suppresses and relieves cough symptoms.

Another objective is to provide an herbal composition that consists of easily available herbal ingredients.

Other systems, devices, methods, features, and advantages will be or become apparent to one with skill in the art upon examination of the following drawings and detailed description. It is intended that all such additional compositions, systems, methods, features, and advantages be included within this description, be within the scope of the present disclosure, and be protected by the accompanying claims and drawings.

### DETAILED DESCRIPTION OF THE INVENTION

Aspects of the invention are disclosed in the following description directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the terms “embodiments of the invention,” “embodiments” or “invention” do not require that all embodiments of the invention include the discussed feature, advantage or mode of operation.

The following description directed to the medicinal properties of individual herbs and mixture of herbs is based, at least in part, on the teachings of Traditional Chinese Medicine (“TCM”). As such, the properties attributed to the individual herbs and mixtures of herbs set forth herein are done so in a non-limiting manner. Any evidence to the contrary based on other understandings or changes in understanding regarding TCM, or past and future scientific research will not operate to affect the operability of embodiments set forth and claimed herein, as will be readily recognized by one having ordinary skill in the art.

The invention is due, in part, to the unexpected finding that administration of the present invention's composition relieves hot cough, a diagnosable condition in TCM. If the patient's pulse speed is more than 80 times a minute, combined with cough symptoms, the condition is diagnosed as a hot cough. Hot cough can also be diagnosed by looking at the color of the tongue coating. Yellow tongue coating red tongue coating indicates hot cough. The color of the sputum also indicates cold or hot cough. Yellow sputum or dark green sputum indicates hot cough. Lastly, if the patient hot sputum symptoms accompanied by symptoms of think nasal discharge or nasal congestion, the patient is suffering from hot cough. The present invention's composition is at least therapeutically effective in treating hot cough and dry cough.

For patients with dry cough, they are usually presented with tiredness, sleepiness, fatigue, weakness, lack of energy, and lack of concentration. This is also a typical symptom of the sequelae of COVID-19. For patients with dry cough, replenishing qi and blood, and restoring physical strength will improve immunity and reduces dry cough symptoms. Even when the symptoms of dry cough are cured, it will take at least three months for the patient to regain his previous physical strength. To completely eliminate dry cough, TCM should aim to regulate the spleen and stomach, strengthen the kidneys and replenish deficiency, moisten the lungs and replenish qi, strengthens the heart and replenish the yang, replenish Qi and blood. Especially for patients who are physically weak, accompanied by loss of appetite, or even eating less, they should focus on replenishing Qi. For patients who have developed chronic pneumonia or Cor pulmonale and rely on oxygen tanks, TCM should aim to increase the regeneration capacity of alveolar cells and get rid of oxygen cylinder supply as soon as possible.

The composition of the present invention consists of the following herbal ingredients mixed in varying weight percentages: Shi Gao, Almonds, Hai Fu Shi, Chuan Bei Mu, Tian Zhu Huang, Ban Xia, Yu Xing Cao, Tian Kui Zi, Gan Cao, Tian Nan Xing, Chai Hu, Huang Qin, Zhi Shi, Bai Shao, Chen Pi, Kuan Dong Hua, Zi Wan, Sheng Jiang. The herbal ingredients aforementioned combine to create a synergy to suppress cough symptoms, improve the circulation of Qi and blood, and balances the forces of Yin and Yang. The present disclosure provides a novel mixture of the ingredients, including unique processing, extraction, and purification methods for optimizing efficacy of the herbal ingredients. The effectiveness of the composition is at least partially dependent on the desired means of preparation and administering the composition.

The individual herbal ingredients are efficacious for nourishing the lung, regulating water in the physical body to reduce or eliminate phlegm. Those skilled in the art will recognize that TCM teaches that the lung is the primary organ that regulates the qi around the body. If the lung is obstructed, so is the qi that circulates in the body. The lung serves four functions of the qi, ascending, descending, inwards and outwards. The lung is an important organ to regulate both qi and blood.

According to the theories of TCM, the spleen produces phlegm and the lung stores phlegm. Cough, profusion of sputum and lung qi reversal occurs when the spleen does not properly dissolve dampness. The herbal composition works to improve balance between wet and dry to reduce and eliminate phlegm.

In a TCM clinical research setting, the composition of the present invention was administered to patients with hot cough and dry cough, about 95%to 98%of the patients experience the relief of cough symptoms within two to three weeks. In one study, a retiree was experiencing symptoms of cough, dry mouth, yellow sputum, constipation and pain in the ribs. The patient tried western medicine without experiencing relief. The patient, at an old age, is experiencing lower immunity to viruses and cold. The patient's pulse indicated weak lung meridian. The patient was administered 14 bottles of the composition, 2 bottles a day, and the symptoms were relieved.

In another case study, a TCM patient was diagnosed with pulmonary fibrosis and pneumonia. The patient was suffering from non-stop cough, and severe cough at night. The patient underwent TCM pulse diagnosis and found that the patient had weak lung meridian, weak kidney meridian and a strong stomach pulse. The patient was administered with a total of 15 bottles of the composition, 2 bottles a day, and the symptoms were relieved.

### Gypsum (Shi Gao)

One herbal ingredient is Gypsum, known as Shi Gao in mandarin. Shi Gao is categorized as heat-clearing and fire-purging herbs, used in TCM to clear heat, sedates fire, relieves irritability and quenches thirst, clears lung heat, clears stomach heat and channel, clears heat and promotes healing from sores, burns, and eczema. Gypsum has low solubility than other herbs so it is common to use in larger dosage. However, if gypsum is administered orally, the dosage is lowered since solubility is no longer applicable.

Shi Gao reduces heat due to stroke, dyspnea in the heart, and asthma, wheezing and sore throat. The pharmacological effects of Shi Gao is antipyretic, immunostimulant, and shown to facilitate the passage of bile, shorten coagulation time, promote diuresis, and reduce spontaneous intake of water in laboratory mice. According to one report, 200 patients with high fever were treated with an herbal decoction (120 grams of Shi Gao, 3 grams of Ma Huang, and 3 grams of Guizhi. Of 200 patients, 181 showed a reduction in body temperature.

### Almonds

Another ingredient is almonds. It is known in TCM, coughing produces a thundering sound, laryngeal numbness, lowering of breath, and produces shortness of breath. decoction of almonds can moisten the heart and lungs, moisten the sound and Qi; remove lung heat and cure the above symptoms. Jiao wind and dryness can relieve chest and diaphragm Qi reversal, and moisturize the large intestine for qi secretion, cough with cold and heat, long-term lung Qi problem.

### Pumex Preparata (Hai Fu Shi)

Another herbal ingredient is Pumex Preparata, known as Haifushi in mandarin. Haifushi is categorized as phlegm-resolving herb, used in TCM for clearing the lung, dissolving phlegm, softens hardness and dissipate nodules, regulates water circulation to treat dysuria.

### Fritilaria Cirrhosa (Chuan Bei Mu)

Another herbal ingredient is Fritilaria Cirrhosa, known as Chuanbeimu, in mandarin. Chuanbeimu is categorized as a Phlegm-resolving and coughing/wheezing relieving herb, used in TCM to dissolve phlegm and stops coughing, clear heat and to disperse nodules. Chuanbeimu is cool and sweet, moistens the lung and dissolves phlegm. It is mostly used for chronic dry cough with scanty difficult-to expectorate sputum, either with or without blood. Chuanbeimu has a marked antitussive effect in rats. The maximum effect was reached 30 minutes after oral administration of herbs, and the effect lasted up to 2 hours.

In a clinical study, equal portions of Chuanbeimu and Kuan Dong Hua were ground into powder and given to children for the treatment of whooping cough in children. The dosage was 1 gram per day for infants under 1 year of age, and adjusted accordingly for children over 1 year of age. Out of 56 cases, 51 showed complete recovery, 4 showed moderate improvement, and 1 had no response.

### Concretio Silicea Bambusae (Tian Zhu Huang)

Another herbal ingredient is Concretio Silicea Bambusae, known as Tian Zhu Huang in mandarin. Tian Zhu Huang is categorized as heat clearing, phlegm dissolving, and ceasing of heart convulsions, used in TCM to reduce convulsions due to heat, stroke from phlegm accumulation and infantile convulsions. Tian Zhu Huang exhibited analgesic effects in mice.

### Pinellia Ternata (Ban Xia)

Another ingredient is Pinellia Ternata, known as Ban Xia in mandarin. Ban Xia is categorized as a phlegm-resolving and coughing/wheezing-relieving herb, used in TCM for drying dampness, dissolving phlegm, redirecting abnormally rising stomach qi, relieves nausea and vomiting, dissipates nodules and disperses stagnation, and treats sores, skin ulcerations and carbuncles. Ban Xia is acrid, warm, and drying, suitable for entering the spleen and lung to eliminate phlegm accumulations.

Qin Ban Xia, is a TCM herbal medicine made from soaking Ban Xia in water and cooked with alumen to reduce its acrid and drying nature, is more suitable for infants, the elderly, and weaker patients who have accumulation of dampness and phlegm. “Zhu Li Ban Xia,” processed with Zhu Li a TCM herbal medicine processed with Zhu Li (Succus Bambusae) to greatly reduce its warm and drying nature, suitable for nausea caused by stomach heat, cough with yellow copious sputum from lung heat, or post-stroke conditions with hot phlegm.

### Herba Houttuyniae (Yu Xing Cao)

Another ingredient is Herba Houttuyniae, known as Yu Xing Cao in mandarin. Yu Xing Cao is categorized as heat-clearing, toxin-eliminating, herb, used in TCM for lung abscess, sore skin lesions from heat and toxins, and promotes normal urination. Yu Xing Cao is acrid and cool, dispels heat and toxins, and drains abscesses and pus. In TCM, it is used to treat lung infections, lung abscesses with coughing, or vomiting with mucus and blood. Sometimes Yu Xing Cao is used with Zhi Mu, Zhe Bei Mu, and Sang Bai Pi to treat cough caused by lung heat. Clinical applications of the herb include pneumonia, acute or chronic bronchitis and enteritis.

In one clinical study, 190 patients with chronic bronchitis were divided into two groups and treated with houttuynium. In the first group the study reported a 78%rate of success with intramuscular injection. In the second group, the study reported a 76.7%success rate from oral ingestion of houttuynium.

### Muskroot (Tian Kui Zi)

Another herbal ingredient is Muskroot, known as Tiankuizi in mandarin. Tiankuizi is categorized as heat-clearing and toxin-eliminating herb, used in TCM for clearing heat and eliminating toxins, dispersion of stagnation and reduces swelling. Tiankuizi resolves and reduces masses characterized by the presence of heat and toxins.

### Radix Glycyrrhizae (Gan Cao)

Another herbal ingredient is Radix Flycyrrhizae, known as Gan Cao in mandarin. Gan Cao is categorized as spleen-tonifying, lung moistening, and a pain-relieving herb, used in TCM to benefit Qi, to treat spleen and stomach deficiencies, stops cough and pain. Gan Cao is neutral in property and mild in action. It treats coughing and wheezing of various etiologies, including cold or heat, and deficiency or excess, with or without phlegm. Gan Cao is also used to treat epigastric, abdominal, musculoskeletal muscle pain and cramps. Gan Cao's pharmacological effects include mineralocorticoid, glucocorticoid, anti-inflammatory, antiarrhythmic, immunologic, gastrointestinal, antispasmodic, antitoxin, hepatoprotective, antitussive and expectorant, analgesic, antibiotic, and antihyperlipidemic.

In one study, 8 out of 9 patients with declining pituitary function were treated successfully by 2 to 3 months of taking 15 to 30 grams of Gan Cao and 6 grams of Radix Ginseng, and for severe symptoms, an additional 10 grams of Radix Aconiti Lateralis Preparata. For those with elevated blood pressure, the dosage of Gan Cao was reduced by 50%and 10 grams of Poria, 10 grams of Cortex Eucommiae, 10 grams of Fuctus Schisandrae Chinensis were added.

In another study, 55 patients with pulmonary tuberculosis were treated with an herbal decoction three times daily for 30 to 90 days. There was significant improvement in 23 patients and some improvement in 32 patients. Rhizoma Arisaematis (Tian Nan Xing)

Another herbal ingredient is Rhizoma Arisaematis, known as Tian Nan Xing in mandarin. Tian Nan Xing has the properties of bitter, acrid, and warm. It enters the meridian channels of liver, lung, and spleen. Tian Nan Xing is categorized as phlegm-resolving, wind-dispelling herb, used in TCM to dry dampness, relieve convulsions, dissipate nodules, and relieve pain. Tian Nan Xing is extremely potent in its function to dry dampness and dissolve phlegm. It is sometimes combined with Fructus Trichosanthis to treat cough and yellow sputum from lung heat. Tian Nan Xing's pharmacological effects are antiseizure and anticonvulsant, sedative, and antineoplastic.

In one study, 105 patients with cervical cancer were treated with a 78%rate of effectiveness using both oral and topical applications of Tian Nan Xing. The treatment protocol was to use a decoction of the herb, starting with 15 g/day and gradually increasing to 45 g/day. In addition to oral administration, local application was given, utilizing various dosage forms such as powder, suppository, and local injection.

### Radix Bupleuri (Chai Hu)

Another herbal ingredient is Radix Bupleuri, known as Chai Hu in mandarin. Chai Hu is categorized as a wind-heat releasing herb, used in TCM to harmonize the exterior and the interior, relieve qi stagnation in the liver, and lift the Yang Qi. Chai Hu is effective in treating patients who exhibit both exterior and interior symptoms such as alternate spells of chills and fever, fullness and distention of the chest, poor appetite, nausea and vertigo. Chai Hu is acrid and aromatic, it has ascending and dispersing characteristics. It is commonly used to unblock liver qi stagnation and spread liver qi. Clinical manifestations of liver qi stagnation include emotional distress, fullness and distention of the chest, hypochondrium, irritability, headache, eye disorders, breast swelling and pain, irregular menstruation, menstrual cramps and jaundice.

Because of its ascending nature, Chai Hu is commonly used to treat prolapse of internal organs caused by yang or qi deficiency. Clinical applications include prolapse of the rectum and uterus with shortness of breath, fatigue, hypermenorrhea, polyuria and other symptoms of yang and qi deficiency. Chai Hu's pharmalogical effects include analgesic and antipyretic, sedative, anti-inflammatory, hepatoprotective, cholagogic, antihyperlipidemic, immunostimulant, and antibiotic.

### Radix Scutellariae (Huang Qin)

Another herbal ingredient is Radix Scutellariae, known as Huang Qin in mandarin. Huang Qin is categorized as a heat-clearing and dampness-drying herb, used in TCM to treat damp-heat disorders (fever, poor appetite, nausea, red tongue coating, yellow tongue coating, rapid and slippery pulse), heat in the middle jiao, clear heat and eliminate toxins, cools blood and stops bleeding.

In one study, 268 patients with infectious or chronic hepatitis were treated with Huang Qin through oral administration and intramuscular injection. The duration of tr4eatment was 1 month for both oral therapy and injection therapy. The effective rate was 63.6% for patients with infectious hepatitis and 73.3% for chronic hepatitis.

### Frutus Aurantii Immaturus (Zhi Shi)

Another herbal ingredient is Frutus Aurantii Immaturus, known as Zhi Shi in mandarin. Zhi Shi is categorized as a qi-regulating herb, used in TCM to break up stagnant qi, resolves accumulation, dissolves distention. Gastrointestinal disorders caused by stagnant qi in the middle jiao is characterized by indigestion, epigastric and abdominal fullness, distention, belching, and foul breath. Zhi Shi is one of the most potent qi-regulating herbs. It breaks up stagnant qi to relief gastrointestinal disorder symptoms.

### Radix Paeoniae Alba (Bai Shao)

Another herbal ingredient is Radix Paenoniae Alba, known as Bai Shao in mandarin. Bai Shao is categorized as a blood-tonifying herb, used in TCM to nourish blood and preserve Yin, nourish liver to calm liver yang and liver wind, and softens the liver and relieve pain. Chronic deficiency of Liver blood results in deficiencies of the ren (conception) and chong (thoroughfare) channels. The symptoms may include irregular menstruation, dysmenorrhea, uterine bleeding, and gestational and postpartum disorders. Bai Shao nourishes blood, softens the liver and is an important herb to regulate menstruation and alleviate pain.

In one study, 85 patients with intestinal cramps and spasms were treated with one dose of an herbal decoction daily. Most patients reported complete remission of spasms and cramps without recurrence. The formula contained Bai Shai, Gui Zhi, Gan Cao, and Mu Gua.

### Dried Mandarin Peel (Chen Pi)

Another herbal ingredient is dried mandarin peel, known as Chenpi in mandarin. Chenpi is categorized as a Qi-regulating herb, used in TCM to regulate Qi, adjusts the middle Jiao, regulates spleen and stomach Qi, dissolves phlegm, and relieve cough.

### Coltsfoot Flower (Kuan Dong Hua)

Another herbal ingredient is Coltsfoot flower, known as Kuan Dong Hua in mandarin. Kuan Dong Hua is categorized as a coughing/wheezing-releasing-herb. Kuan Dong Hua is used in TCM to dissolve phlegm, stops cough due to lung cold, lung heat, or lung yin deficiency. It is most effective for treating cough due to cold attacking the lung, or chronic cough with blood-streaked sputum. When combined with appropriate herbs, Kuan Dong Hua treats upper respiratory infection, influenza, and a wide range of coughing disorders, including cold or hot, exterior or interior, and excess or deficient conditions. In a clinical study, extract of Kuan Dong Hua (equivalent to 6 grams of herb) given three times daily provided marked relief in treating 36 asthma patients. The preparation, however, was ineffective for treating acute and severe onset of an asthma attack.

### Tetarian Aster (Zi Wan)

Another herbal ingredient is Tetarian Aster, known as Ziwan in mandarin. Ziwan is categorized as a cough/wheezing-releasing-herb, used in TCM to dissolve phlegm and stops cough due to wind-cold, wind-heat, or lung deficiency. Ziwan is warm but not hot, and moistening. Zi Wan has an antibiotic influence against E coli, Bacillus dysenteriae, Bacillus proteus, Salmonella typhi, Bacillus paratyphosus, Pseudomonas aeruginosa, Vibrio cholerae, and some dermatophytes and influenza viruses. Zi wan has show antineoplastic effects, preliminary inhibitory effects on the growth of cancer cells in mice.

### Ginger (Sheng Jiang)

Another herbal ingredient is Ginger, known as Sheng Jiang in mandarin. Sheng Jiang is categorized as a wind-cold releasing herb, used in TCM to release the exterior and induce perspiration, warms the middle jiao and alleviates vomiting, warms the lung and stops coughing, and eliminating toxins. Sheng Jiang's pharmacological effects include gastrointestinal, cardiovascular, and antibiotic. In one study, Sheng Jiang preparation was injected in 113 patients to evaluate its effect on arthritis and lower back pain. The injections were administered to pressure points daily or every other day, for 3 to 5 treatments per course. The rate of effectiveness was 83%.

The extract of each of the above identified herbs is prepared and decocted by traditional procedures known in the art which includes boiling, filtering, and forming concentrates.

In a preferred embodiment of the present invention, a method to brew an herbal beverage product has the following steps: the grinding together of all these aforementioned ingredients to obtain a dispersion of finely ground brewing particles, adding an aqueous solution to the ground particles, heating the aqueous solution from about room temperature to about boiling temperature, reduction of the aqueous liquid by simmering, filtering the aqueous liquid to obtain the first extract, adding an aqueous solution to the remaining solids of the first extract, heat the aqueous solution containing solids from the extract from about room temperature to about boiling temperature, reduction of the aqueous solution contain the first extract by simmering, filtering the aqueous solution containing solids from the first extract to obtain the second extract, combining both extracts to create a third extract, reducing the third extract with a condensing machine.

To form the composition of the present invention in one embodiment, the extracted concentrate of each herb is mixed according to the following measurements: Shi Gao, about 15 to 25 percent by dry weight, Yu Xing Cao, about 11 to 19 percent by dry weight, Chuan Bei Mu, about 11 to 19 percent by dry weight, Tian Kui Zi, about 11 to 19 percent by dry weight, Tian Zhu Huang, about 11 to 19 percent by dry weight, Hai Fu Shi, about 7 to 13 percent by dry weight, Gan Cao, about 7 to 13 percent by dry weight.

To form the composition of the present invention in another embodiment, the extracted concentrate comprises: Shi Gao, Almonds, Hai Fu Shi, Chuan Bei Mu, Tian Zhu Huan, Zhi Ban Xia.

The composition of the present invention is for ingestion in the form of a tea, liquid beverage, tablet, capsule, powder, or soup. Conventionally known methods known in the art are used to prepare the composition in the different forms. The form in which the composition is prepared does not significantly affect its efficacy. The ideal form of the composition is an herbal tea. For mild symptoms, ½ of the preferred quantity should be consumed in the morning, and ½ of the preferred quantity should be consumed in the evening. The ideal form of the composition, in an herbal tea, is to mix with 1 part water and 1 part composition, heated over the countertop or microwave, and consumed after a meal. The composition should be taken daily on a regular basis to obtain maximum benefits, until the cough symptom alleviates.

While a preferred embodiment of the invention has been described and illustrated for purposes of clarity and example, it should be understood that many changes, substitutions and modifications to the described embodiment will be apparent to those having skill in the art in light of the foregoing disclosure without departing from the scope and spirit of the present invention which is defined by the claim which will follow.

What is claimed is:

1. A composition for provisioning an herbal tea for suppression of cough symptoms, comprising: Shi Gao, Yu Xing Cao, Chuan Bei Mu, Tian Kui Zi, Tian Zhu Huang, Hai Fu Shi, Gan Cao.

2. The composition of claim 1, wherein the composition includes at least one member selected from the group comprising of: Almonds, Tian Zhu Huang, Ban Xia, Tian Nan Xing, Chai Hu, Huang Qin, Zhi Shi, Bai Shao, Chen Pi, Kuan Dong Hua, Zi Wan, Ginger.

3. The composition of claim 1, wherein the composition is administered in at least one of the following ways: oral, topical, suppository, intravenous, intradermic, intramuscular, and intraperitoneal.

4. The composition of claim 1, wherein the composition includes at least one member selected from the group comprising of: a liquid beverage, an aqueous beverage, a tablet, an orally disintegrating tablet, a pill, a dripping pill, a lozenge, a capsule, a cream, a granule, and a suppository.

5. The composition of claim 1, wherein the composition consists of a plurality of herbal ingredients prepared through processing methods, extraction methods, and purification methods.

6. The composition of claim 1, wherein the processing methods include at least one member selected from the group consisting of: purification, grinding, pulverizing, soaking, heating, evaporation, compressing, washing, steaming, extracting, roasting, herb frying, salt frying, honey frying, wine frying, earth frying, vinegar frying, calcining and drying.

7. A composition for provisioning an herbal tea for suppression of cough symptoms, comprising: Shi Gao, Almonds, Hai Fu Shi, Chuan Bei Mu, Tian Zhu Huang, Ban Xia.

8. The composition of claim 7, wherein the composition includes at least one member selected from the group comprising of: Yu Xing Cao, Tian Kui Zi, Gan Cao, Tian Nan Xing, Chai Hu, Huang Qin, Zhi Shi, Bai Shao, Chen Pi, Kuan Dong Hua, Zi Wan, Ginger.

9. The composition of claim 7, wherein the composition is administered in at least one of the following ways: oral, topical, suppository, intravenous, intradermic, intramuscular, and intraperitoneal.

10. The composition of claim 7, wherein the composition includes at least one member selected from the group comprising of: a liquid beverage, an aqueous beverage, a tablet, an orally disintegrating tablet, a pill, a dripping pill, a lozenge, a capsule, a cream, a granule, and a suppository.

11. The composition of claim 7, wherein the composition consists of a plurality of herbal ingredients prepared through processing methods, extraction methods, and purification methods.

12. The composition of claim 7, wherein the processing methods include at least one member selected from the group consisting of: purification, grinding, pulverizing, soaking, heating, evaporation, compressing, washing, steaming, extracting, roasting, herb frying, salt frying, honey frying, wine frying, earth frying, vinegar frying, calcining and drying.

13. A method for preparing an herbal cough relief beverage composition comprising the steps of:
Adding together herbal ingredients comprising Shi Gao, Yu Xing Cao, Chuan Bei Mu, Tian Kui Zi, Tian Zhu Huang, Hai Fu Shi, Gan Cao. Grinding the herbal ingredients to obtain a dispersion of finely ground brewing particles. Add an aqueous solution to the ingredients. Heat the aqueous solution from about room temperature to about boiling temperature [symbol] Reduction of the aqueous liquid by simmering. Filtering the aqueous liquid to obtain the extract.

14. The method of claim 13, wherein the processing methods include at least one member selected from the group consisting of: purification, grinding, pulverizing, soaking, heating, evaporation, compressing, washing, steaming, extracting, roasting, herb frying, salt frying, honey frying, wine frying, earth frying, vinegar frying, calcining and drying.

15. The method of claim 13, wherein the herbal ingredients further comprise of at least one ingredient from the group comprises of: Almonds, Tian Zhu Huang, Ban Xia, Tian Nan Xing, Chai Hu, Huang Qin, Zhi Shi, Bai Shao, Chen Pi, Kuan Dong Hua, Zi Wan, Ginger.

16. The method of claim 13, wherein the ingredients further comprise at least one additive selected from the group consisting of caloric sweetener, non-caloric sweetener, flavoring, coloring, preservative, caffeine, guarana, vitamin, dietary mineral, amino acid, protein, taurine, omega-3 fatty acid, essential fatty acid, polyunsaturated fat, monounsaturated fat, and medium chain triglyceride.

17. The method of claim 13, wherein the brewing ingredient comprises a summation of one or more members selected from the group consisting of tea leaves, herb, spice, dried fruit, dried vegetable, and cacao.

18. The method of claim 13, wherein the brewing ingredient comprises at least one polysaccharide selected from the group consisting of inulin, chicory root extract, acacia gum, ester gum, gellan gum, guar gum, locust bean gum, tara gum, xanthan gum, agar, alginin, arrowroot, carrageenan, dextrin, maltodextrin, methyl cellulose, pectin, polydextrose, stevia, stevia extract, tapioca, oligosaccharide, starch, thickener, and vegetable gum.

19. The method of claim 13, wherein the brewing ingredient comprises a summation of one or more members selected from the group consisting of non-dried fruit and non-dried vegetable.
